(19) United States
(12) Patent Application Publication
(10) Pub. No.: US 2025/0058137 A1
(43) Pub. Date: Feb. 20, 2025

(54) System and Method For Enhancing Cartilage-Specific Collagen Formation Using Stem Cells and A Support Garment Having A Composite Fabric Containing Embedded Semiconductor and Carbon Nanoparticles

(71) Applicant: Incrediwear Holdings, Inc., Chico, CA (US)

(72) Inventor: JACKSON CORLEY, Chico, CA (US)

(73) Assignee: Incrediwear Holdings, Inc., Chico, CA (US)

(21) Appl. No.: 18/800,189

(22) Filed: Aug. 12, 2024

Related U.S. Application Data
(63) Continuation-in-part of application No. 18/451,800, filed on Aug. 17, 2023, now Pat. No. 12,096,809.
(60) Provisional application No. 63/520,336, filed on Aug. 17, 2023.
(60) Provisional application No. 63/387,453, filed on Dec. 14, 2022.
(60) Provisional application No. 63/387,486, filed on Dec. 14, 2022.

(51) Int. Cl. A61N 2/00; A61K 35/28; A61N 5/06; A61P 19/02

(52) U.S. Cl. CPC A61N 2/002; A61K 35/28; A61N 2/008; A61N 5/0613; A61P 19/02; A61N 2005/0645; A61N 2005/0659

(57) ABSTRACT

A system for enhancing cartilage-specific collagen formation in a human body includes stem cells configured for injection into a region of the body, and a support garment configured for contact with the region of the body. The support garment includes a composite fabric having embedded semiconductor nanoparticles configured to release negative ions responsive to body heat generating a micro electromagnetic field in the region of the body. The support garment also includes embedded carbon nanoparticles configured to release infrared rays responsive to the body heat in the region of the body. A method for treating osteoarthritis in the human body includes: injecting the stem cells into the region of the body, and placing the support garment having the composite fabric in contact with the region of the body to form the micro electromagnetic field and generate the infrared rays in the region of the body.

### CROSS REFERENCE TO RELATED APPLICATION

This application is a continuation-in-part of U.S. application Ser. No. 18/451,800, filed Aug. 17, 2023, and also claims priority from U.S. provisional application Ser. No. 63/520,336, filed Aug. 17, 2023. Each of these applications is incorporated herein by reference.

### FIELD

This disclosure relates to medical systems and medical treatment methods that employ stem cells to treat humans. More particularly, this disclosure relates to a system and method for enhancing cartilage-specific collagen using stem cells and a support garment having a composite fabric containing semiconductor nanoparticles and carbon nanoparticles. The system and method are particularly adapted to the treatment of osteoarthritis.

### BACKGROUND

Adipose derived stem cells (ADSCs) can be used in different medical therapeutic procedures to treat various medical conditions in human bodies. For example, adipose derived stem cells can be used to aid tissue regeneration in the treatment of osteoarthritis. Osteoarthritis can be identified by the degeneration of articular cartilage, and is also characterized by joint function impairment and pain. In the treatment of osteoarthritis, adipose stem cells in the form of mesenchymal stem cells (and/or stromal cells) have shown promise in regenerative medicine when applied to articular cartilage. These types of stem cells can be obtained from liposuction waste, and can be applied to a particular region of the body using injection.

The present disclosure is directed to a system and method for enhancing cartilage-specific collagen using stem cells injected into a region of the body in combination with a support garment made from a composite fabric configured for contact with the region and having embedded semiconductor nanoparticles and embedded carbon nanoparticles.

### SUMMARY

A system for enhancing cartilage-specific collagen formation in a human body includes a plurality of stem cells configured for injection into a region of the body, and a support garment configured for contact with the region of the body. The support garment comprises a composite fabric having embedded semiconductor nanoparticles and embedded carbon nanoparticles. In addition, the support garment can be configured as an item of apparel, such as a brace, or as a medical specific device, such as a bandage or wrap. The system enhances formation of type-2 cartilage, limiting potential scaring from type-3 or type-4 collagen formation.

The composite fabric includes a plurality of woven threads constructed in any weaving pattern. In a first embodiment, each thread comprises a combination of at least three components in an integrated structure including: a base material comprising a plurality of fibers, a plurality of semiconductor nanoparticles compounded with the base material, and a plurality of carbon nanoparticles compounded with the base material. In the first embodiment, additional components can also be included in the integrated structure such as jade fibers. In a second embodiment, the composite fabric includes at least three separate threads including a base thread, a semiconductor thread, and a carbon thread twisted together into a yarn. The twisted threads can also include other types of threads such as jade threads. In both thread embodiments, other separate threads, such as elastic threads or jade threads, can also be incorporated into the woven structure.

In both thread embodiments, the semiconductor nanoparticles are configured to release negative ions responsive to a transdermal effect from heat in the region of the body flowing through the skin into the composite fabric for generating a micro electromagnetic field in the region of the body. In addition, the carbon nanoparticles are configured to release infrared waves responsive to the transdermal effect from the heat in the region of the body flowing into the composite fabric. The micro electromagnetic field in combination with the infrared rays increases cellular vibration of the stem cells, and increases blood circulation in the region of the body contacted by the composite fabric. In addition, the micro electromagnetic field in combination with the infrared rays decreases inflammation, tissue damage, stiffness and pain, in the region of the body contacted by the composite fabric.

A method for treating osteoarthritis in a human body includes the steps of injecting a plurality of stem cells into a region of the body, and placing a support garment comprising a composite fabric having embedded semiconductor nanoparticles and embedded carbon nanoparticles in contact with the region of the body. The method also includes the step of releasing negative ions to form a micro electromagnetic field in the region of the body using a first transdermal effect from body heat activating the semiconductor nanoparticles to release the negative ions and form the micro electromagnetic field. The method also includes the step of releasing infrared rays using a second transdermal effect from body heat activating the carbon nanoparticles to release the infrared rays. The action of the negative ions, micro electromagnetic field and infrared rays increases blood circulation in the region of the body and reduces inflammation.

### BRIEF DESCRIPTION OF THE DRAWINGS

FIG. 1 is a schematic drawing of a system for enhancing cartilage-specific collagen formation in a human body comprising stem cells and a support garment having a composite fabric containing embedded semiconductor nanoparticles and embedded carbon nanoparticles;

FIG. 2A is an enlarged schematic cross-sectional view illustrating a first embodiment thread of the composite fabric having an integrated structure;

FIG. 2B is an enlarged schematic cross-sectional view illustrating a second embodiment thread of the composite fabric wherein separate threads are twisted together into a yarn;

FIG. 3 is a schematic diagram illustrating operational characteristics of the composite fabric in which negative ions and infrared rays are released responsive to body heat;

FIG. 4 is a schematic diagram illustrating operational characteristics of the composite fabric in which blood circulation in a region of the body contacted by the composite fabric is increased and inflammation as well as tissue damage, stiffness and pain are decreased;

FIG. 5A is a schematic diagram illustrating free radical molecules attacking a body cell;

FIG. 5B is a schematic diagram illustrating an effect of the negative ions released by the semiconductor nanoparticles of the composite fabric combining with free radical molecules to form stable molecules;

FIG. 6A is a schematic diagram illustrating a body part in the form of a knee joint having osteoarthritis;

FIG. 6B is a schematic diagram illustrating the knee joint having osteoarthritis covered by the support garment made from the composite fabric;

FIG. 7 is a schematic diagram illustrating different configurations of the support garment made from the composite fabric;

FIG. 8 is a schematic view of a support garment in the form of a medical wrap; and

FIG. 9 is a flow diagram illustrating steps in a method for treating osteoarthritis in a human body using the support garment having the composite fabric containing embedded semiconductor nanoparticles and embedded carbon nanoparticles.

### DETAILED DESCRIPTION

The term “support garment” means a garment configured to provide a level of support for a region of the body or a body part. The term “osteoarthritis” means a degenerative joint disease, which occurs most frequently in the hands, hips, and knees, wherein the cartilage within a joint begins to break down and the underlying bone begins to change. The term “adipose derived stem cells” means stem cells derived from adipose tissue. The term “woven threads” means interweaving multiple threads to form a pattern that holds together. The term “elastic thread” means a thread that exhibits stretch and recovery characteristics. For example, an elastic thread can be stretched to 100-200% of its unstretched length responsive to a tension force and then return to the unstretched length when the tension force is removed. The term “a first transdermal effect” refers to negative ions and infrared radiation flowing from the composite fabric through the skin into the region of the body. The term “a second transdermal effect” refers to body heat flowing through the skin into the composite fabric.

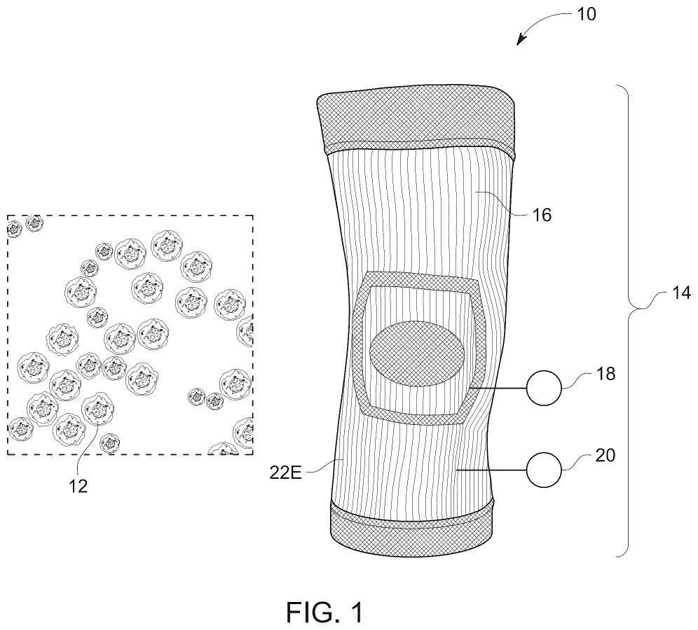

Referring to FIG. 1, a system 10 for enhancing cartilage-specific collagen formation in a human body is illustrated. The system 10 includes a plurality of stem cells 12 configured for injection into a region of the body, and a support garment 14 configured for contact with the region of the body. The support garment 14 comprises a composite fabric 16 having embedded semiconductor nanoparticles 18 and embedded carbon nanoparticles 20.

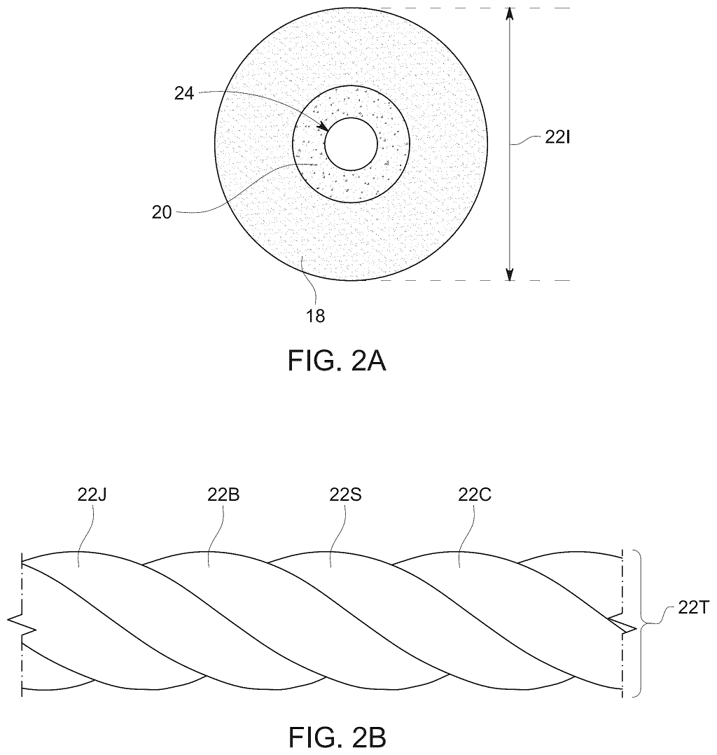

Referring to FIGS. 2A and 2B, the composite fabric 16 (FIG. 1) includes a plurality of threads 221 (FIG. 2A) or 22T (FIG. 2B) woven together in any weaving pattern. In a first embodiment shown in FIG. 2A, an integrated thread 221 comprises a combination of at least three components in an integrated structure including: a base material 24 comprising a plurality of fibers, a plurality of semiconductor nanoparticles 18 compounded with the base material 24, and a plurality of carbon nanoparticles 20 compounded with the base material 24. In FIG. 2A, the integrated thread 221 is shown with a layered structure for illustrative purpose, however, in actual practice the three components can comprise a unitary structure. Exemplary materials for the base material 24 include cotton, nylon and polyester in fiber form. The semiconductor nanoparticles 18 and the carbon nanoparticles 20 can be made using a process such as high temperature sintering (800-1000 degrees C.) of raw materials, such as germanium pellets for the semiconductor nanoparticles 18 and carbonized charcoal for the carbon nanoparticles 20, followed by grinding and filtering into nanoparticles having a selected size range, with from 1 to 100 nm being representative. The semiconductor nanoparticles 18 and the carbon nanoparticles 20 can then be compounded with the fibers of the base material 24 using a process such as doping or polymerization or resin formation. U.S. application Ser. No. 18/451,800, which was previously incorporated herein by reference, further describes suitable fabrication processes for making the integrated thread 221, as well as relative concentrations of the materials.

In a second embodiment, the composite fabric 16 (FIG. 1) includes twisted threads 22T formed as a yarn using at least three separate threads including a base thread 22B, a semiconductor thread 22S, and a carbon thread 22C twisted together into a yarn. The twisted threads 22T can also include other types of threads such as a jade thread 22J. U.S. application Ser. No. 18/451,800, which was previously incorporated by reference, further describes suitable fabrication processes for making the twisted threads 22T. In addition to the twisted threads 22T (or the integrated threads 221), the composite fabric 16 (FIG. 1) can also include other types of threads in the woven structure, such as elastic threads 22E (FIG. 1).

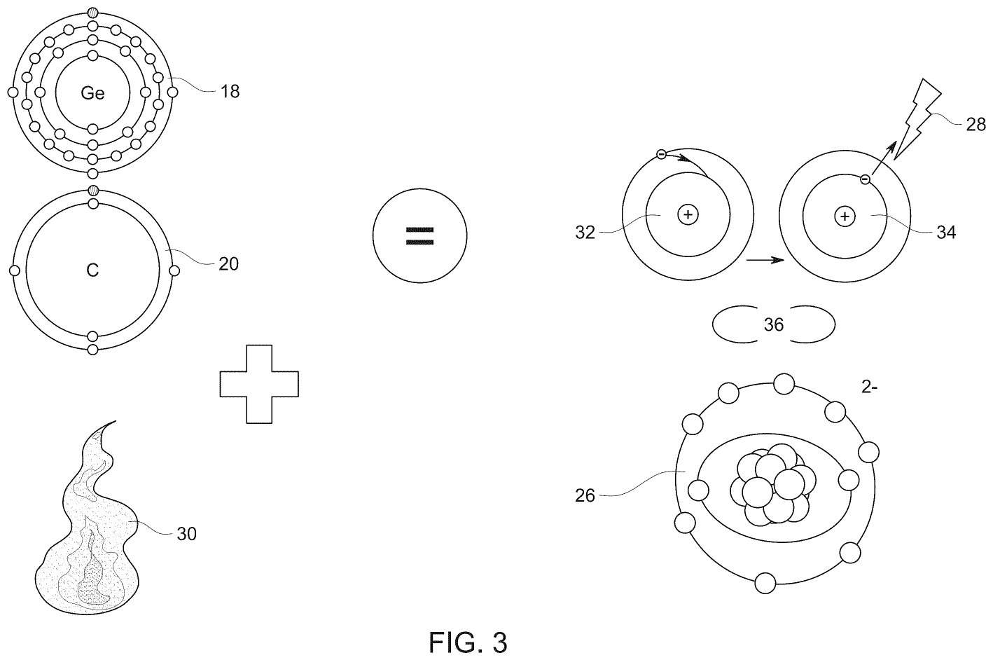

Referring to FIG. 3, the semiconductor nanoparticles 18 are configured to release negative ions 26 responsive to a transdermal effect from body heat 30 in the region of the body contacted by the composite fabric 16. The semiconductor nanoparticles 18 have a concentration in the composite fabric 16 (FIG. 1) sufficient to generate a low intensity micro electromagnetic field 36 in the region of the body contacted by the composite fabric 16. A preferred material for the semiconductor nanoparticles 18 comprises germanium having a size range of 1-20 nm. Alternately, other semiconductor materials, such as silicon, silicon carbide, and selenium, as well as compound semiconductor materials, can be used to construct the composite fabric 16 (FIG. 1). A representative intensity of the micro electromagnetic field 36 can be from 1730-2405 ions/cm³. For treating osteoarthritis, a threshold intensity of the micro electromagnetic field 36 to reach therapeutic benefit preferably comprises at least 2000 ions/cm³. The intensity of the micro electromagnetic field 36 on a region of the body can be measured using a commercial micro electromagnetic field sensor. One factor that affects the intensity of the micro electromagnetic field 36 is the concentration of the semiconductor nanoparticles 18 in the integrated thread 221 and in the twisted thread 22T. A representative concentration range of the semiconductor nanoparticles 18 as a weight % of the total weight of the integrated thread 221 can be from 5% to 80%.

As also shown in FIG. 3, the carbon nanoparticles 20 are configured to release infrared waves 28 responsive to the transdermal effect from the body heat 30 in the region of the body contacted by the composite fabric 16. In FIG. 3, the infrared waves 28 are shown transitioning from a ground state 32 to an excited state 34 responsive to the body heat 30. A preferred material for the carbon nanoparticles 20 comprises charcoal, such as bamboo charcoal having a size range of 10-45 nm. Other suitable forms of carbon include graphite carbon and carbon black. A representative wavelength range of the infrared waves 38 can comprise mid infrared to far infrared, with from 1300 nm to 3000 nm being exemplary.

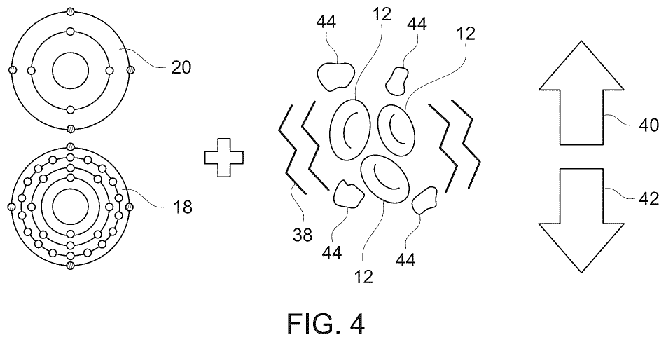

Referring to FIG. 4, operational characteristics of the composite fabric 16 (FIG. 1) are illustrated. In particular, the semiconductor nanoparticles 18 increase cellular vibration of body cells 44 in the region of the body contacted by the composite fabric 16, including the stem cells 12, as indicated by the vibration waves 38. This increase in vibration of the body cells 44 and the stem cells 12 also increases blood circulation, and blood flux, in the region of the body contacted by the composite fabric 16, as indicated by up-arrow 40. At the same time, inflammation, tissue damage, stiffness and pain, are decreased in the region of the body contacted by the composite fabric 16, as indicated by down-arrow 42. In addition, the increased blood circulation provides O2 and nutrients in the region of the body contacted by the composite fabric 16. The increased blood circulation also pushes out prostaglandins or other wastes in the region of the body contacted by the composite fabric 16 and optimizes the healing and cell growth functions of the stem cells 12 (FIG. 1).

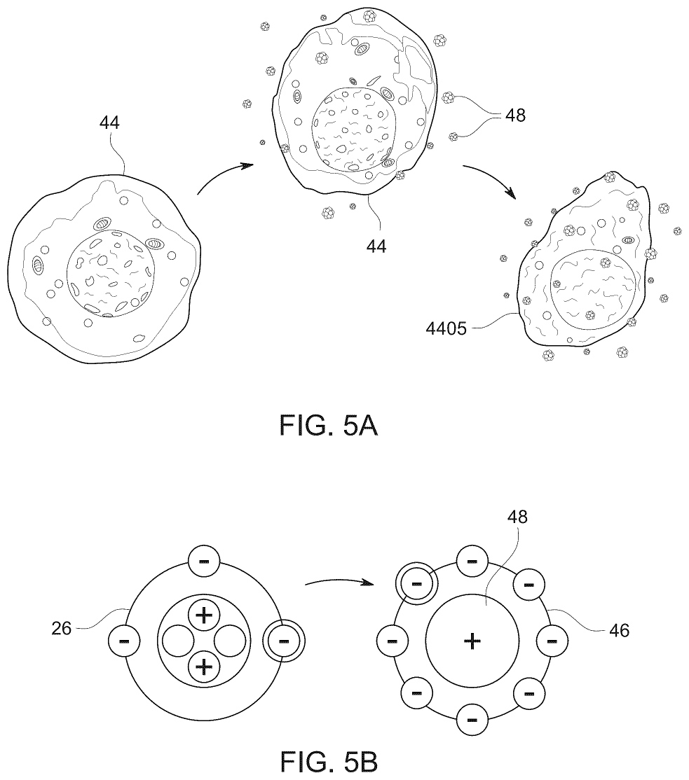

Referring to FIGS. 5A and 5B, further operational characteristics of the composite fabric 16 (FIG. 1) are illustrated. FIG. 5A is a schematic diagram illustrating free radical molecules 48 attacking a body cell 44 and forming an oxidative stressed body cell 440S. FIG. 5B is a schematic diagram illustrating the effect of the negative ions 26 released by the semiconductor nanoparticles 18 of the composite fabric 16 on the oxidative stressed body cell 44OS. In particular, the negative ions 26 can include 2 protons and 3 electrons for attracting and combining with positive free radical molecules 48, such as the oxidative stressed body cell 44OS, to form stable molecules 46.

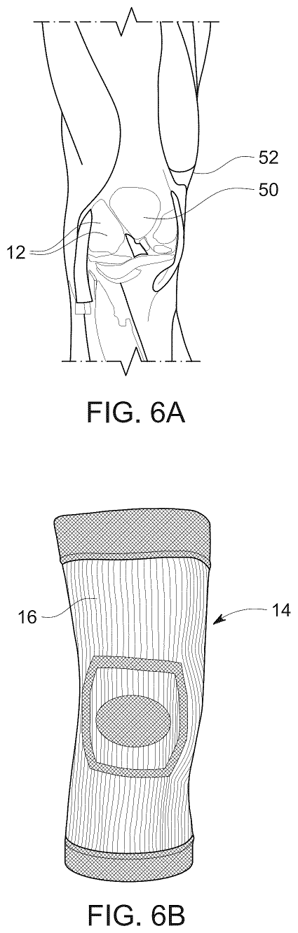

Referring to FIGS. 6A and 6B, further operational characteristics of the composite fabric 16 (FIG. 1) are illustrated. In FIG. 6A, a knee joint 50 has osteoarthritis and has been injected with stem cells 12. In FIG. 6B, the support garment 14 has been placed in contact with the skin 52 proximate to the knee joint 50. The composite fabric 16 increases blood circulation in the knee joint 50, substantially as previously described. In addition, the composite fabric 16 decreases inflammation, decreases tissue damage, reduces stiffness and pain, and pushes out prostaglandins in the knee joint 50, substantially as previously described.

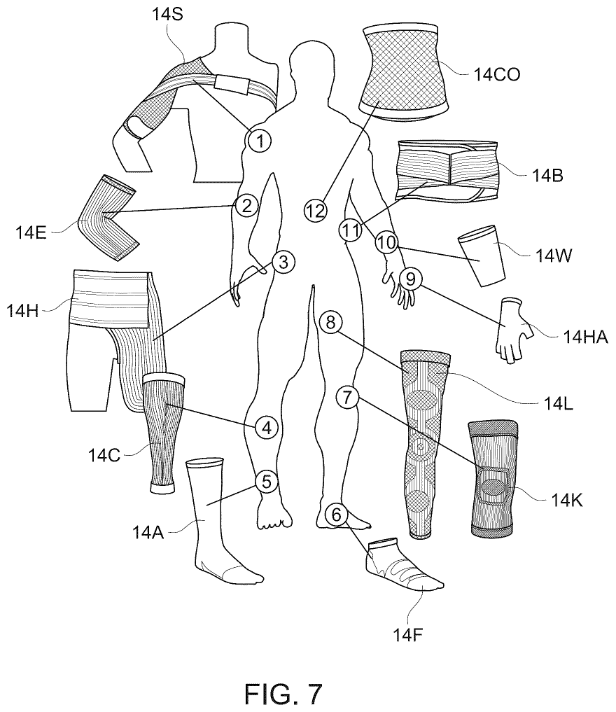

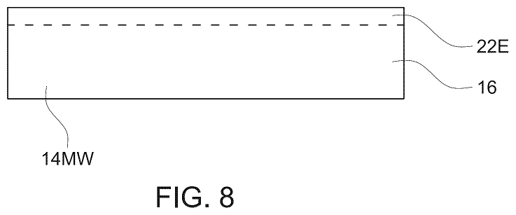

Referring to FIG. 7, different embodiments for the support garment 14 are illustrated. These embodiments can include: support garment (shoulder) 14S, support garment (elbow) 14E, support garment (hip) 14H, support garment (calf) 14C, support garment (ankle) 14A, support garment (feet) 14F, support garment (knee) 14K, support garment (leg) 14L, support garment (hand) 14HA, support garment (wrist) 14W, support garment (back) 14B, and support garment (core) 14CO. In each of the embodiments, the support garment 14 can be sized and shaped to fit snuggly around the selected region of the body or body part, such that contact can be made with the skin in the region or body part for transferring body heat 30 to the semiconductor nanoparticles 18 and the carbon nanoparticles 20 using a transdermal effect. As shown in FIG. 8, the support garment 14 can also comprise a medical wrap 14MW made from the composite fabric 16 and containing a plurality of elastic threads 22E, similar to an ACE™ brand elastic bandage manufactured by 3-M Corporation.

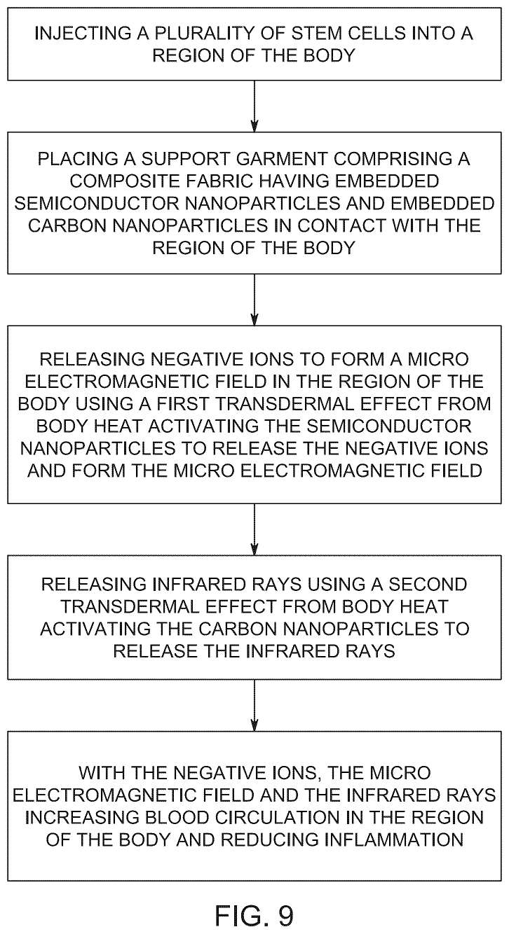

Referring to FIG. 9, steps in a method for treating osteoarthritis in a human are illustrated.
Example: A proof of concept study was conducted at the request of Applicant by Duranti et al., which has been cited in Applicant's Information Disclosure Statement. The purpose of the study was to determine the effects of germanium embedded fabric manufactured by Applicant under the trademark Incrediwear®, on the chondrogenic differentiation of adipose derived stem cells used in the treatment of osteoarthritis.

Based upon the results shown in Duranti et al., a cartilage-specific collagen type-II expression substantially increased in cells treated with Incrediwear® material and maintained in a differentiation medium (Diff+Incred) when compared to both its untreated relative control (Diff) and Basal conditions. Incrediwear®-material treatment enhances formation of good type-2 collagen, limiting potential scaring from type-3 or type-4 collagen formation.

The results in Duranti et al., also show treatment of ADSCs (Adipose-Derived Stem Cells) with Incrediwear® material induces modulation of certain of the main genetic chondrogenesis markers, as well as inducing modifications in cell morphology indicative of chondrogenic differentiation and cartilage regeneration COL2A1 demonstrating upregulation in its expression rate following the described treatment schedule with Incrediwear® material in differentiation medium cultures compared to untreated controls in differentiation medium. Those results also show that treatment with Incrediwear® material can help slow osteoarthritic changes by upregulation of COL2A1.

The results in Duranti et al. also show that the marker for runt-related transcription factor 2 (the RUNX2 marker) exhibited a substantial increase in Incrediwear®-material-treated cultures under differentiation medium conditions.

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope.

What is claimed is:

1. A system for enhancing cartilage-specific collagen formation in a human body comprising:
a plurality of stem cells configured for injection into a region of the body; and
a support garment configured for contact with the region of the body, the support garment comprising a composite fabric having embedded semiconductor nanoparticles and embedded carbon nanoparticles,
the semiconductor nanoparticles configured to release negative ions responsive to heat in the region of the body flowing into the composite fabric for generating a micro electromagnetic field in the region of the body,
the carbon nanoparticles configured to release infrared waves responsive to the heat in the region of the body flowing into the composite fabric,
the composite fabric comprising a plurality of woven threads, each thread comprising a combination of at least three components in an integrated structure including a base material comprising a plurality of fibers, a plurality of semiconductor nanoparticles compounded with the base material, and a plurality of carbon nanoparticles compounded with the base material.

2. The system of claim 1 wherein the semiconductor nanoparticles comprise germanium and the carbon nanoparticles comprises charcoal.

3. The system of claim 1 wherein each thread includes jade fibers.

4. The system of claim 1 wherein the micro electromagnetic field has an intensity of 1730-2405 ions/cm³.

5. The system of claim 1 wherein the stem cells comprise adipose derived stem cells.

6. The system of claim 1 wherein the base material comprises a material selected from the group consisting of cotton, nylon and polyester.

7. The system of claim 1 wherein the support garment comprises a garment selected from the group consisting of a shoulder support garment, an elbow support garment, a hip support garment, a calf support garment, an ankle support garment, a foot support garment, a knee support garment, a leg support garment, a hand support garment, a wrist support garment, a back support garment, and a core support garment.

8. The system of claim 1 wherein a concentration range of the semiconductor nanoparticles as a weight % of a total weight of each thread is from 5% to 80%.

9. The system of claim 1 wherein the composite fabric includes a plurality of elastic threads.

10. A system for enhancing cartilage-specific collagen formation in a human body comprising:
a plurality of stem cells configured for injection into a region of the body; and
a support garment configured for contact with the region of the body, the support garment comprising a composite fabric having embedded semiconductor nanoparticles and embedded carbon nanoparticles,
the semiconductor nanoparticles configured to release negative ions responsive to heat in the region of the body flowing into the composite fabric for generating a micro electromagnetic field in the region of the body,
the carbon nanoparticles configured to release infrared waves responsive to the heat in the region of the body flowing into the composite fabric,
the composite fabric comprising a plurality of woven threads comprising at least three separate threads including a base thread, a semiconductor thread containing the semiconductor nanoparticles, and a carbon thread containing the carbon nanoparticles twisted together into a yarn.

11. The system of claim 10 wherein the semiconductor nanoparticles comprise germanium and the carbon nanoparticles comprises charcoal.

12. The system of claim 10 wherein the woven threads include a plurality of jade threads.

13. The system of claim 10 wherein the micro electromagnetic field has an intensity of 1730-2405 ions/cm³.

14. The system of claim 10 wherein the stem cells comprise adipose derived stem cells.

15. The system of claim 10 wherein the base thread comprises a material selected from the group consisting of cotton, nylon and polyester.

16. The system of claim 10 wherein the support garment comprises a garment selected from the group consisting of a shoulder support garment, an elbow support garment, a hip support garment, a calf support garment, an ankle support garment, a foot support garment, a knee support garment, a leg support garment, a hand support garment, a wrist support garment, a back support garment, and a core support garment.

17. The system of claim 10 wherein a concentration range of the semiconductor nanoparticles as a weight % of a total weight of each woven thread is from 5% to 80%.

18. The system of claim 10 wherein the composite fabric includes a plurality of elastic threads.

19. A method for treating osteoarthritis in a human body comprising:
injecting a plurality of stem cells into a region of the body;
placing a support garment comprising a composite fabric having embedded semiconductor nanoparticles and embedded carbon nanoparticles in contact with the region of the body, the semiconductor nanoparticles configured to release negative ions responsive to body heat flowing into the composite fabric for generating a micro electromagnetic field in the region of the body, the carbon nanoparticles configured to release infrared waves into the region of the body responsive to the body heat flowing into the composite fabric;
releasing negative ions to form a micro electromagnetic field in the region of the body using a first transdermal effect from the body heat activating the semiconductor nanoparticles to release the negative ions and form the micro electromagnetic field; and
releasing infrared rays using a second transdermal effect from the body heat activating the carbon nanoparticles to release the infrared rays into the region of the body.

20. The method of claim 19 wherein the negative ions, the micro electromagnetic field and the infrared rays increase blood circulation in the region of the body and reduce inflammation.

21. The method of claim 19 wherein the composite fabric comprises a plurality of woven threads, each thread comprising a combination of at least three components in an integrated structure including a base material comprising a plurality of fibers, a plurality of semiconductor nanoparticles compounded with the base material, and a plurality of carbon nanoparticles compounded with the base material.

22. The method of claim 19 wherein the composite fabric comprises a plurality of woven threads, each thread comprising at least three separate threads including a base thread, a semiconductor thread containing the semiconductor nanoparticles, and a carbon thread containing the carbon nanoparticles twisted together into a yarn.

23. The method of claim 19 wherein the semiconductor nanoparticles comprise germanium and the carbon nanoparticles comprises charcoal.

24. The method of claim 19 wherein the micro electromagnetic field has an intensity of 1730-2405 ions/cm³.

25. The method of claim 19 wherein the stem cells comprise adipose derived stem cells.

26. The method of claim 19 wherein the support garment comprises a garment selected from the group consisting of a shoulder support garment, an elbow support garment, a hip support garment, a calf support garment, an ankle support garment, a foot support garment, a knee support garment, a leg support garment, a hand support garment, a wrist support garment, a back support garment, and a core support garment.
